SEQUENCE LISTING (12 sequences)

SEQ ID NO: 1 - DNA, 33 bp, Artificial
  a primer used in the amplification of a region encoding a replication protein and a replication origin of pUB110 plasmid
atagagctcacgaagtcgagatcagggaatgag

SEQ ID NO: 2 - DNA, 36 bp, Artificial
  a primer used in the amplification of a region encoding a replication protein and a replication origin of pUB110 plasmid
gcgagatctctcgtcttcctaagcatccttcaatcc

SEQ ID NO: 3 - DNA, 178 bp, Artificial
  a base sequence of a replication origin of pUB110 plasmid
cttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaataga

SEQ ID NO: 4 - DNA, 1005 bp, Artificial
  a base sequence of a region encoding a replication protein of pUB110 plasmid
atgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtag

SEQ ID NO: 5 - protein, 334 aa, Artificial
  an amino acid sequence of a replication protein (RepA) of pUB110 plasmid
MGVSFNIMCPNSSIYSDEKSRVLVDKTKSGKVRPWREKKIANVDYFELLHILEFKKAERVKDCAEILEYKQNRETGERKLYRVWFCKSRLCPMCNWRRAMKHGIQSQKVVAEVIKQKPTVRWLFLTLTVKNVYDGEELNKSLSDMAQGFRRMMQYKKINKNLVGFMRATEVTINNKDNSYNQHMHVLVCVEPTYFKNTENYVNQKQWIQFWKKAMKLDYDPNVKVQMIRPKNKYKSDIQSAIDETAKYPVKDTDFMTDDEEKNLKRLSDLEEGLHRKRLISYGGLLKEIHKKLNLDDTEEGDLIHTDDDEKADEDGFSIIAMWNWERKNYFIKE

SEQ ID NO: 6 - DNA, 498 bp, Artificial
  a base sequence of a DNA fragment including a multiple cloning site used in the construction of a shuttle plasmid pLK1-MCS of the present invention
atacccgggcatgattttaagggggttagcagatgcataagtttaatttttttgttaaaaaatattaaactttgtgttttttttaacaaaatatattgataaaaataataatagtgggtataattaagttgttagagaaaacgtataaattagggataaactatggaacttatgaaatagattgaaatggtttatctgttaccccgtatcaaaatttaggaggttagtttaaacctgcagagatctctcgaggcggccgcgtcgactctagacccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgagctcata

SEQ ID NO: 7 - DNA, 738 bp, Artificial
  a base sequence of an erythromycin resistance gene
atgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataa

SEQ ID NO: 8 - DNA, 861 bp, Artificial
  a base sequence of an ampicillin resistance gene
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 9 - DNA, 615 bp, Artificial
  a base sequence of a replication origin of pUC19 plasmid
tcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacg

SEQ ID NO: 10 - DNA, 5014 bp, Artificial
  a base sequence of a shuttle plasmid pLK1-MCS of the present invention
catgattttaagggggttagcagatgcataagtttaatttttttgttaaaaaatattaaactttgtgttttttttaacaaaatatattgataaaaataataatagtgggtataattaagttgttagagaaaacgtataaattagggataaactatggaacttatgaaatagattgaaatggtttatctgttaccccgtatcaaaatttaggaggttagtttaaacctgcagagatctctcgaggcggccgcgtcgactctagacccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgagctcacgaagtcgagatcagggaatgagtttataaaataaaaaaagcacctgaaaaggtgtctttttttgatggttttgaacttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagagatcctagcagcacgccatagtgactggcgatgctgtcggaatggacgatcaaattccccgtaggcgctagggacctctttagctccttggaagctgtcagtagtatacctaataatttatctacattccctttagtaacgtgtaactttccaaatttacaaaagcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgccattctttgctgtttcgatttttatgatatggtgcaagtcagcacgaacacgaaccgtcttatctcccattatatctttttttgcactgattggtgtatcatttcgtttttctttttatcccgcaagaggcccggcagtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggg

SEQ ID NO: 11 - DNA, 39 bp, Artificial
  a base sequence of a primer used in the amplification of a region encoding a secondary alcohol dehydrogenase through PCR reaction
cacaggcctatgaaaggttttgcaatgctaggtattaat

SEQ ID NO: 12 - DNA, 39 bp, Artificial
  a base sequence of a primer used in the amplification of a region encoding a secondary alcohol dehydrogenase through PCR reaction
atatctagattatttatcacctctgcaaccacagccacc

(19) United States
(12) Patent Application Publication
(10) Pub. No.: US 2016/0230179 A1
(43) Pub. Date: Aug. 11, 2016

(54) SHUTTLE PLASMID REPLICABLE IN BOTH CLOSTRIDIA AND ESCHERICHIA COLI

(71) Applicant: GS CALTEX CORPORATION, Seoul (KR)

(72) Inventor: Sang-Hyun LEE, Daejeon (KR)

(21) Appl. No.: 15/015,626

(22) Filed: Feb. 4, 2016

(30) Foreign Application Priority Data
Feb. 10, 2015 (KR) 10-2015-0020510

(51) Int. Cl. C12N 15/74; C12N 15/70

(52) U.S. Cl. CPC C12N 15/74; C12N 15/70

(57) ABSTRACT

Disclosed is a shuttle plasmid replicable in both Clostridium and Escherichia coli comprising: a base sequence of a first replication origin replicable in Escherichia coli; and a base sequence of a second replication origin derived from pUB 110 plasmid.

### CROSS REFERENCE TO RELATED APPLICATION

This application claims the benefit of Korean Patent Application No. 10-2015-0020510, filed on Feb. 10, 2015, which is hereby incorporated by reference in its entirety into this application.

### BACKGROUND

1. Technical Field

The present invention relates to a shuttle plasmid replicable in both Clostridium and Escherichia coli.

2. Description of the Related Art

Microorganisms of the genus Clostridium are Gram-positive bacteria. They are obligate anaerobes capable of producing spores and have a variety of activity as biocatalysts. Recently, as the full genomes of some microorganisms of the genus Clostridium have recently been sequenced, it is expected that studies into Clostridium will contribute to the production of biofuels from renewable biomass and development of methods for interpreting and preventing pathogenic mechanisms of some microorganisms of the genus Clostridium. Although Clostridium is important industrially and medicinally, research of Clostridium has been extremely limited due to absence of useful genetic engineering techniques. Accordingly, there is a need for development of effective host/plasmid systems useful for developing Clostridium strains having an effectively high metabolic activity.

On the other hand, in order to replicate a plasmid in acetobutylicum and/or Escherichia coli, shuttle plasmids such as pMTL500E (Minton N P, Oultram J D. 1988. Host: vector systems for gene cloning in Clostridium. Microbiological Sciences 5: 310-315), pIM1 (Mermelstein L D, Papoutsakis E T. 1993. In vivo methylation in Escherichia coli by the Bacillus subtilis phage phi 3TI methyltransferase to protect plasmids from restriction upon transformation of Clostridium acetobutylicum ATCC 824. Appl Environ Microbiol 59:1077-1081.), pJIR418 (Sloan J, Warner T A, Scott P T, Bannam T L, Berryman D I, Rood J I. 1992. Construction of a sequenced Clostridium perfringens-Escherichia coli shuttle plasmid. Plasmid 27:207-219.), and pCB 102 (Fox M E, Lemmon M J, Mauchline M L, Davis T O, Giaccia A J, Minton N P, Brown J M. 1996. Anaerobic bacteria as a delivery system for cancer gene therapy: in vitro activation of 5-fluorocytosine by genetically engineered clostridia. Gene therapy 3: 173-178.), and the like have been developed. The Clostridial replication origins (origins of replication) are derived from pAM β1, pIMP13, pIP404, and pCB102, respectively. However, no plasmid is used industrially since their segregational stability is very low under culture conditions with no antibiotic present. Namely, since use of antibiotics may deteriorate economic feasibility of microorganisms in terms of industrial application or can cause problems related to environmental stability, in order for plasmids to be used industrially, segregational stability of the plasmids in a medium containing no antibiotics should be guaranteed. The shuttle plasmids such as pMTL500E, pIM1, pJIR418, pCB102, and the like do not possess the requisite segregational stability.

The present inventors have endeavored to find a novel plasmid having excellent segregational stability in a culture medium containing no antibiotics and capable of replication in Clostridium acetobutylicum, and constructed plasmids including specific replication origins and regions encoding replication proteins, and the like which are found to be replicable in both Clostridium and Escherichia coli and have high segregational stability. The present invention is based on this finding.

### BRIEF SUMMARY

It is an aspect of the present invention to provide a shuttle plasmid replicable in both Clostridium and Escherichia coli.

In accordance with one aspect of the present invention, there is provided a shuttle plasmid replicable in both Clostridium and Escherichia coli, including: a base sequence of a first replication origin replicable in Escherichia coli; and a base sequence of a second replication origin derived from pUB110 plasmid.

The shuttle plasmid may be replicable in both Clostridium and Escherichia coli.

### BRIEF DESCRIPTION OF THE DRAWINGS

The above and other aspects, features, and advantages of the present invention will become apparent from the detailed description of the following embodiments in conjunction with the accompanying drawings, in which:

FIG. 1 shows a genetic map of cryptic plasmid pUB110 derived from Staphylococus aureus;

FIG. 2 shows a process for constructing a shuttle plasmid pLK1-MCS from pUB110 cryptic plasmid and pMTL500E plasmid including a base sequence of a replication origin of pUC19 plasmid;

FIG. 3 shows segregational stability of a shuttle plasmid pLK1-MCS of the present invention; and

FIG. 4 shows a process for constructing a recombinant plasmid in which a recombinant gene is cloned so that acetone is converted into isopropanol by using the shuttle plasmid of the present invention.

### EXPLANATION OF SEQ ID NO

SEQ ID NO: 1 and SEQ ID NO: 2 are base sequences of two primers used in the amplification of a region encoding a replication protein and a replication origin of pUB110 plasmid.

SEQ ID NO: 3 is a base sequence of a replication origin of pUB110 plasmid.

SEQ ID NO: 4 is a base sequence of a region encoding a replication protein of pUB110 plasmid.

SEQ ID NO: 5 is an amino acid sequence of a replication protein (RepA) of pUB110 plasmid.

SEQ ID NO: 6 is a base sequence of a DNA fragment including a multiple cloning site used in the construction of a shuttle plasmid pLK1-MCS of the present invention.

SEQ ID NO: 7 is a base sequence of an erythromycin resistance gene.

SEQ ID NO: 8 is a base sequence of an ampicillin resistance gene.

SEQ ID NO: 9 is a base sequence of a replication origin of pUC19 plasmid.

SEQ ID NO: 10 is a base sequence of a shuttle plasmid pLK1-MCS of the present invention.

SEQ ID NO: 11 and SEQ ID NO: 12 are base sequences of primers used in the amplification of a region encoding a secondary alcohol dehydrogenase through PCR reaction.

### DETAILED DESCRIPTION

The present invention relates to a shuttle plasmid replicable in both Clostridium and Escherichia coli including:

a base sequence of a first replication origin replicable in Escherichia coli; and

a base sequence of a second replication origin derived from pUB110 plasmid.

In addition, the present invention relates to a shuttle plasmid replicable in both Clostridium and Escherichia coli including:

a base sequence of pMTL500E plasmid including a replication origin of pUC19 plasmid (pUC origin) and ampicillin and erythromycin antibiotic resistance genes; and

a base sequence of a base sequence of a replication protein and a replication origin derived from pUB110 plasmid (pUB110 origin).

Further, the present invention relates to a method for constructing a transformed microorganism including:

preparing a shuttle plasmid of the present invention; and

introducing the shuttle plasmid into a microorganism.

Furthermore, the present invention relates to a transformed microorganism including a shuttle plasmid of the present invention.

Furthermore, the present invention relates to a method for producing a culture including:

culturing a transformed microorganism including a shuttle plasmid of the present invention; and

harvesting the culture.

Hereinafter, embodiments of the present invention will be described in detail.

### Base Sequence of a First Replication Origin Replicable in Escherichia coli

The shuttle plasmid of the present invention includes a base sequence of a first replication origin replicable in Escherichia coli. The base sequence of the first replication origin may be preferably a base sequence of a replication origin derived from pUC19 plasmid. Namely, the base sequence of the first replication origin may be derived from pUC (pUC origin). In addition, the base sequence of a replication origin derived from pUC19 plasmid may be derived from pMTL500E plasmid.

### Base Sequence of a Second Replication Origin Derived from pUB110 Plasmid

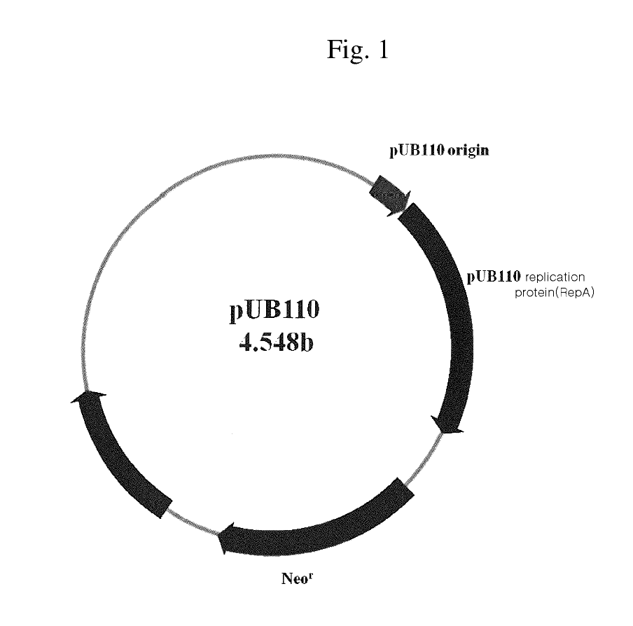

The shuttle plasmid of the present invention includes a base sequence of a second replication origin derived from pUB110 plasmid. pUB110 plasmid is a cryptic plasmid derived from Staphylococcus aureus, and the genetic map thereof is depicted in FIG. 1. The base sequence of a replication origin derived from pUB110 plasmid may be a base sequence of SEQ ID NO: 3. In addition, the base sequence may have 70% or more, preferably 80% or more, more preferably 90% or more homology with SEQ ID NO: 3 and maintain functionality of the replication origin.

### Base Sequence Encoding a Replication Protein Derived from pUB110 Plasmid

The shuttle plasmid of the present invention may include a base sequence encoding a replication protein and derived from pUB110 plasmid. The base sequence encoding the replication protein may be a base sequence of SEQ ID NO: 4. The base sequence may have 70% or more, preferably 80% or more, more preferably 90% or more homology with SEQ ID NO: 4 and encode a protein maintaining the functionality of the replication protein. On the other hand, the amino acid sequence of the replication protein may be an amino acid sequence of SEQ ID NO: 5. The amino acid sequence may have 70% or more, preferably 80% or more, more preferably 90% or more homology with SEQ ID NO: 5 and maintain the functionality of the replication protein.

### First Antibiotic Resistance Gene

The shuttle plasmid of the present invention may include a first antibiotic resistance gene capable of being expressed in Escherichia coli and serving as a selective marker in Escherichia coli. The first antibiotic resistance gene is expressed in Escherichia coli and serves as a selective marker. Preferably, the first antibiotic resistance gene is an ampicillin antibiotic resistance gene.

### Second Antibiotic Resistance Gene

The shuttle plasmid of the present invention may include a second antibiotic resistance gene capable of being expressed in Clostridium and serving as a selective marker in Clostridium. The second antibiotic resistance gene is expressed in Clostridium, preferably Clostridium acetobutylicum and serves as a selective marker. Preferably, the second antibiotic resistance gene is an erythromycin antibiotic resistance gene.

However, it is not intended that the first antibiotic resistance gene is limited to the ampicillin antibiotic resistance gene, or the second antibiotic resistance gene is limited to erythromycin antibiotic resistance gene. For example, the first antibiotic resistance gene and the second antibiotic resistance gene may be identical. The first antibiotic resistance gene may be any antibiotic resistance gene so long as such gene can serve as a selective marker in Escherichia coli. The second antibiotic resistance gene may be any antibiotic resistance gene so long as such gene can serve as a selective marker in Clostridium.

### Shuttle Plasmid of the Present Invention

The shuttle plasmid of the present invention is a shuttle plasmid replicable in both Clostridium and Escherichia coli including:

a base sequence of a first replication origin replicable in Escherichia coli; and

a base sequence of a second replication origin derived from pUB110 plasmid.

Further, the shuttle plasmid of the present invention may be a shuttle plasmid replicable in both Clostridium and Escherichia coli including:

a base sequence of a first replication origin replicable in Escherichia coli;

a base sequence of a second replication origin derived from pUB110 plasmid;

a base sequence encoding a replication protein derived from pUB110 plasmid;

a first antibiotic resistance gene expressed in Escherichia coli; and

a second antibiotic resistance gene expressed in Clostridium.

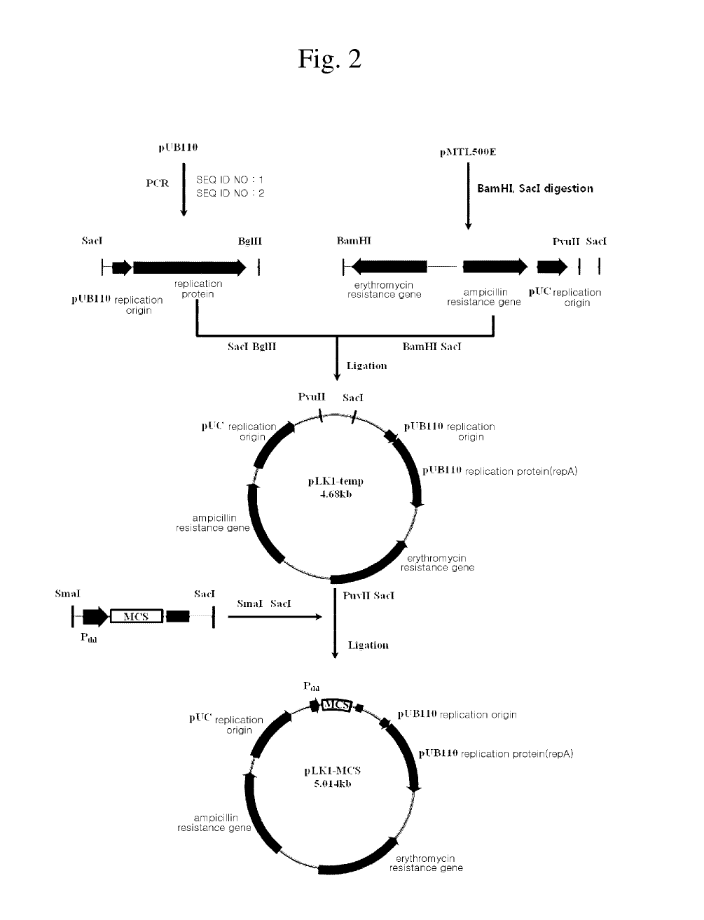

The shuttle plasmid of the present invention may include a base sequence of SEQ ID NO: 7, or a base sequence having 80% or more, preferably 90% or more, more preferably 95% or more homology with SEQ ID NO: 7 so long as the base sequence maintains replication capability in both Escherichia coli and Clostridium, and expression and selection marker capability of antibiotic resistance genes in both Escherichia coli and Clostridium. Further, the shuttle plasmid of the present invention may be the pLK1-MCS. In addition, the shuttle plasmid of the present invention can be easily constructed using pUB110 cryptic plasmid and pMTL500E plasmid, wherein pMTL500E plasmid includes a replication origin of pUC19 plasmid, the first antibiotic resistance gene and the second antibiotic resistance gene (FIG. 2).

The shuttle plasmid of the present invention is a shuttle vector replicable in both Clostridium and Escherichia coli. Particularly, the shuttle plasmid has high segregational stability in Clostridium. Therefore, use of the shuttle plasmid of the present invention may allow the shuttle plasmid and a target gene to be recombined, which functions as an operably linked recombinant shuttle plasmid. Further, the shuttle plasmid of the present invention is capable of replication in both Clostridium and Escherichia coli under an environment with no antibiotics and under an environment with antibiotics. Specifically, the shuttle plasmid of the present invention is capable of replication in both Clostridium and Escherichia coli under the environment with no antibiotics, and has high segregational stability, thereby being industrially applicable.

Further, the shuttle plasmid of the present invention has high segregational stability in Clostridium strains, and is sufficiently replicable in a culture medium containing no antibiotics, thereby ensuring stability in a fermentation process or a biotransformation process.

Clostridium may include Clostridium acetobutylicum, Clostridium beijerinckii, Clostridium saccharobutylicum, Clostridium saccharoperbutylacetonicum, Clostridium perfringens, Clostridium tetani, Clostridium difficile, Clostridium butylicum, Clostridium butylicum, Clostridium kluyveri, Clostridium tyrobutylicum or Clostridium tyrobutyricum. Preferably, Clostridium is Clostridium acetobutylicum.

### Method for Constructing Transformed Microorganisms

The present invention provides a method for constructing a transformed microorganism including: preparing a shuttle plasmid of the present invention; and introducing the shuttle plasmid into a microorganism.

After preparing the shuttle plasmid, a foreign gene is cloned to the shuttle plasmid, which is introduced into a microorganism.

Furthermore, the present invention provides a method for producing a transformed microorganism including: preparing a shuttle plasmid of the present invention; cloning a foreign gene into the shuttle plasmid; and introducing the shuttle plasmid into which the foreign gene is cloned into a microorganism.

In addition, the present invention provides a transformed microorganism including the shuttle plasmid of the present invention. The microorganism may be Escherichia coli or Clostridium.

### Method for Producing Culture Products

The present invention provides a method for producing a culture including: culturing a transformed microorganism including the shuttle plasmid of the present invention; and harvesting the culture.

Further, the present invention provides a method for producing a fermented product including: culturing a transformed microorganism including the shuttle plasmid of the present invention; collecting the culture; and harvesting a fermented product produced by the transformed microorganism from the culture.

Further, the present invention provides a method for producing a fermented product including: preparing a shuttle plasmid of the present invention; cloning a foreign gene into the shuttle plasmid; and introducing the shuttle plasmid into a microorganism to construct a transformed microorganism; culturing the transformed microorganism; collecting the culture; and harvesting a fermented product from the culture.

The microorganism may be Escherichia coli or Clostridium. The culture contains fermented products produced by the transformed microorganism. The fermented products may be fermented products originally produced by the microorganism or fermented products produced by the foreign gene. For example, the fermented products may be alcohols, organic acids, ketones, and the like, preferably, alcohols having carbon number of 7 or less, polyhydric alcohols, and the like. For example, the fermented products may be butanol, isopropanol, ethanol, 1,3-propanol, 2,3-butandiol, propionic acid, acetone, and the like, without being limited thereto.

The above and other aspects, features, and advantages of the present invention will become apparent from the detailed description of the following embodiments in conjunction with the accompanying drawings. However, it should be understood that the present invention is not limited to the following embodiments and may be embodied in different ways, and that the embodiments are provided for complete disclosure and thorough understanding of the invention by those skilled in the art. The scope of the invention should be defined only by the accompanying claims and equivalents thereof.

### Materials and Methods

pUB110 plasmid and Clostridium acetobutylicum ATCC 824 were purchased from the US strain depository authority, American Type Culture Collection (ATCC).

### EXPERIMENTAL EXAMPLE 1

Construction of pLK1-MCS Shuttle Plasmid

A region encoding a replication protein and a replication origin of pUB110 plasmid were amplified and obtained using a primer having a base sequence of SEQ ID NO: 1 and a primer having a base sequence of SEQ ID NO: 2 and using pUB110 cryptic plasmid as a template. The base sequence of the replication origin of pUB110 plasmid is the base sequence of SEQ ID NO: 3; the base sequence encoding a replication protein (RepA) is the base sequence of SEQ ID NO: 4; and the amino acid sequence of a replication protein is the amino acid of SEQ ID NO: 5.

100 μl of PCR reaction mixture was prepared by combining 250 μM dNTP, 20 pmol of each primer, 1.5 mM MgCl2, 10 μl of 10× buffer, 100 ng of DNA template, and 1 unit of pfu polymerase. In PCR reaction, the reaction repeated 30 cycles consisting of initial denaturing at 95° C. for 5 minutes, followed by denaturing at 95° C. for one minute, annealing at 58° C. for one minute and then polymerizing at 72° C. for two minutes. PCR reaction in the following Examples was performed in the same manner as above. The amplified DNA fragment was purified on a 1% agarose gel, and then digested with SacI/BglII restriction enzymes to isolate a DNA fragment.

TABLE 1

| | |
|---|---|
| SEQ ID NO: 1 | ATAGAGCTCACGAAGTCGAGATCAGGGAATGA |
| | G |
| SEQ ID NO: 2 | GCGAGATCTCTCGTCTTCCTAAGCATCCTTCA |
| | ATCC |
| SEQ ID NO: 3 | CTTGTTCTTTCTTATCTTGATACATATAGAAA |
| | TAACGTCATTTTTATTTTAGTTGCTGAAAGGT |
| | GCGTTGAAGTGTTGGTATGTATGTGTTTTAAA |
| | GTATTGAAAACCCTTAAAATTGGTTGCACAGA |
| | AAAACCCCATCTGTTAAAGTTATAAGTGACTA |
| | AACAAATAACTAAATAGA |
| SEQ ID NO: 4 | ATGGGGCTTTCTTTTAATATTATGTGTCCTAA |
| | TAGTAGCATTTATTCAGATGAAAAATCAAGGG |
| | TTTTAGTGGACAAGACAAAAAGTGGAAAAGTG |
| | AGACCATGGAGAGAAAAGAAAATCGCTAATGT |
| | TGATTACTTTGAACTTCTGCATATTCTTGAAT |
| | TTAAAAAGGCTGAAAGAGTAAAAGATTGTGCT |
| | GAAATATTAGAGTATAAACAAAATCGTGAAAC |
| | AGGCGAAAGAAAGTTGTATCGAGTGTGGTTTT |
| | GTAAATCCAGGCTTTGTCCAATGTGCAACTGG |
| | AGGAGAGCAATGAAACATGGCATTCAGTCACA |
| | AAAGGTTGTTGCTGAAGTTATTAAACAAAAGC |
| | CAACAGTTCGTTGGTTGTTTCTCACATTAACA |
| | GTTAAAAATGTTTATGATGGCGAAGAATTAAA |
| | TAAGAGTTTGTCAGATATGGCTCAAGGATTTC |
| | GCCGAATGATGCAATATAAAAAAATTAATAAA |
| | AATCTTGTTGGTTTTATGCTGCAACGGAAGTG |
| | ACAATAAATAATAAAGATAATTCTTATAATCA |
| | GCACATGCATGTATTGGTATGTGTGGAACCAA |
| | CTTATTTTAAGAATACAGAAAACTACGTGAAT |
| | CAAAAACAATGGATTCAATTTTGGAAAAAGGC |
| | AATGAAATTAGACTATGATCCAAATGTAAAAG |
| | TTCAAATGATTCGACCGAAAAATAAATATAAA |
| | TCGGATATACAATCGGCAATTGACGAAACTGC |
| | AAAATATCCTGTAAAGGATACGGATTTTATGA |
| | CCGATGATGAAGAAAAGAATTTGAAACGTTTG |
| | TCTGATTTGGAGCAAGGTTTACACCGTAAAAG |
| | GTTAATCTCCTATGGTGGTTTGTTAAAAGAAA |
| | TACATAAAAAATTAAACCTTGATGACACAGAA |
| | GAAGGCGATTTGATTCATACAGATGATGACGA |
| | AAAAGCCGATGAAGATGGATTTTCTATTATTG |
| | CAATGTGGAATTGGGAACGGAAAAATTATTTT |
| | ATTAAAGAGTAG |
| SEQ ID NO: 5 | MGVSFNIMCPNSSIYSDEKSRVLVDKTKSGKV |
| | RPWREKKIANVDYFELLHILEFKKAERVKDCA |
| | ETLEYKQNRETGERKLYRVWFCKSRLCPMCNW |
| | RRAMKHGIQSQKVVAEVIKQKPTVRWLFLTLT |
| | VKNVYDGEELNKSLSDMAQGFRRNIMQYKKIN |
| | KNLVGFMRATEVTINNKDNSYNQHMHVLVCVE |
| | PTYFKNTENYVNQKQWIQFWKKAMKLDYDPNV |
| | KVQMIRPKNKYKSDIQSAIDETAKYPVKDTDF |
| | MTDDEEKNLKRLSDLEEGLHRKRLISYGGLLK |
| | EIHKKLNLDDTEEGDLIHTDDDEKADEDGFSI |
| | IAMWNWERKNYFIKE |

On the other hand, as depicted in FIG. 2, pMTL500E plasmid was cleaved with SacI/BamHI restriction enzymes and the resulting DNA fragment containing an ampicillin resistance gene, an erythromycin resistance gene and a replication origin region of pUC19 plasmid (pUC origin) were purified in a 1% agarose gel, and then, the digested fragment ligated with DNA fragments (SEQ ID NOs: 3 and 4) comprising a replication origin of pUB110 and a replication protein, which was amplified by PCR reaction as mentioned above, digested with SacI/BglII restriction enzymes to construct pLK1-temp. The constructed pLK1-temp was digested with PvuII/SacI restriction enzymes, cloned together with a DNA fragment excised from DNA (SEQ ID NO: 6) by digestion of SmaI/SacI, which include a promoter and a multiple cloning site synthesized by Bioneer Corp. to construct a final pLK1-MCS (SEQ ID NO: 10, FIG. 2). The pLK1-MCS includes a base sequence of the erythromycin resistance gene (SEQ ID NO: 7), a base sequence of the ampicillin resistance gene (SEQ ID NO: 8) and a base sequence of the replication origin of pUC19 plasmid (SEQ ID NO: 9).

The pLK1-MCS (DNA plasmid) was deposited with accession number of KCTC 12755BP at the Korea Research Institute of Bioscience and Biotechnology (BRIBB) on Feb. 4, 2015.

TABLE 2

| | |
|---|---|
| SEQ ID NO: 6 | ATACCCGGGCATGATTTTAAGGGGGTTAGCAG |
| | ATGCATAAGTTTAATTTTTTTGTTAAAAAATA |
| | TTAAACTTTGTGTTTTTTTTAACAAAATATAT |
| | TGATAAAAATAATAATAGTGGGTATAATTAAG |
| | TTGTTAGAGAAAACGTATAAATTAGGGATAAA |
| | CTATGGAACTTATGAAATAGATTGAAATGGTT |
| | TATCTGTTACCCCGTATCAAAATTTAGGAGGT |
| | TAGTTTAAACCTGCAGAGATCTCTCGAGGCGG |
| | CCGCGTCGACTCTAGACCCGGGAATTCACTGG |
| | CCGTCGTTTTACAACGTCGTGACTGGGAAAAC |
| | CCTGGCGTTACCCAACTTAATCGCCTTGCAGC |
| | ACATCCCCCTTTCGCCAGCTGGCGTAATAGCG |
| | AAGAGGCCCGCACCGATCGCCCTTCCCAACAG |
| | TTGCGCAGCCTGAATGGCGAATGGCGCCTGAT |
| | GCGGTATTTTCTCCTTACGCATCTGTGCGGTA |
| | TTTCACACCGAGCTCATA |
| SEQ ID NO: 7 | ATGAACAAAAATATAAAATATTCTCAAAACTT |
| | TTTAACGAGTGAAAAAGTACTCAACCAAATAA |
| | TAAAACAATTGAATTTAAAAGAAACCGATACC |
| | GTTTACGAAATTGGAACAGGTAAAGGGCATTT |
| | AACGACGAAACTGGCTAAAATAAGTAAACAGG |
| | TAACGTCTATTGAATTAGACAGTCATCTATTC |
| | AACTTATCGTCAGAAAAATTAAAACTGAATAC |
| | TCGTGTCACTTTAATTCACCAAGATATTCTAC |
| | AGTTTCAATTCCCTAACAAACAGAGGTATAAA |
| | ATTGTTGGGAGTATTCCTTACCATTTAAGCAC |
| | ACAAATTATTAAAAAAGTGGTTTTTGAAAGCC |
| | ATGCGTCTGACATCTATGTGATTGTTGAAGAA |
| | GGATTCTACAAGCGTACCTTGGATATTCACCG |
| | AACACTAGGGTTGCTCTTGCACACTCAAGTCT |
| | CGATTCAGCAATTGCTTAAGCTGCCAGCGGAA |
| | TGCTTTCATCCTAAACCAAAAGTAAACAGTGT |
| | CTTAATAAAACTTACCCGCCATACCACAGATG |
| | TTCCAGATAAATATTGGAACCTATATACGTAC |
| | TTTGTTTCAAAATGGGTCAATCGAGAATATCG |
| | TCAACTGTTTACTAAAAATCAGTTTCATCAAG |
| | CAATGAAACACGCCAAAGTAAACAATTTAAGT |
| | ACCGTTACTTATGAGCAAGTATTGTCTATTTT |
| | TAATAGTTATCTATTATTTAACGGGAGGAAAT |
| | AA |
| SEQ ID NO: 8 | ATGAGTATTCAACATTTCCGTGTCGCCCTTAT |
| | TCCCTTTTTTGCGGCATTTTGCCTTCCTGTTT |
| | TTGCTCACCCAGAAACGCTGGTGAAAGTAAAA |
| | GATGCTGAAGATCAGTTGGGTGCACGAGTGGG |
| | TTACATCGAACTGGATCTCAACAGCGGTAAGA |
| | TCCTTGAGAGTTTTCGCCCCGAAGAACGTTTT |
| | CCAATGATGAGCACTTTTAAAGTTCTGCTATG |
| | TGGCGCGGTATTATCCCGTATTGACGCCGGGC |
| | AAGAGCAACTCGGTCGCCGCATACACTATTCT |
| | CAGAATGACTTGGTTGAGTACTCACCAGTCAC |
| | AGAAAAGCATCTTACGGATGGCATGACAGTAA |
| | GAGAATTATGCAGTGCTGCCATAACCATGAGT |
| | GATAACACTGCGGCCAACTTACTTCTGACAAC |
| | GATCGGAGGACCGAAGGAGCTAACCGCTTTTT |
| | TGCACAACATGGGGGATCATGTAACTCGCCTT |
| | GATCGTTGGGAACCGGAGCTGAATGAAGCCAT |
| | ACCAAACGACGAGCGTGACACCACGATGCCTG |
| | TAGCAATGGCAACAACGTTGCGCAAACTATTA |
| | ACTGGCGAACTACTTACTCTAGCTTCCCGGCA |
| | ACAATTAATAGACTGGATGGAGGCGGATAAAG |
| | TTGCAGGACCACTTCTGCGCTCGGCCCTTCCG |
| | GCTGGCTGGTTTATTGCTGATAAATCTGGAGC |
| | CGGTGAGCGTGGGTCTCGCGGTATCATTGCAG |
| | CACTGGGGCCAGATGGTAAGCCCTCCCGTATC |
| | GTAGTTATCTACACGACGGGGAGTCAGGCAAC |
| | TATGGATGAACGAAATAGACAGATCGCTGAGA |
| | TAGGTGCCTCACTGATTAAGCATTGGTAA |
| SEQ ID NO: 9 | TCAAAGGATCTTCTTGAGATCCTTTTTTTCTG |
| | CGGGTAATCTGCTGCTTGCAAACAAAAAAACC |
| | ACCGCTACCAGCGGTGGTTTGTTTGCCGGATC |
| | AAGAGCTACCAACTCTTTTTCCGAAGGTAACT |
| | GGCTTCAGCAGAGCGCAGATACCAAATACTGT |
| | TCTTCTAGTGTAGCCGTAGTTAGGCCACCACT |
| | TCAAGAACTCTGTAGCACCGCCTACATACCTC |
| | GCTCTGCTAATCCTGTTACCAGTGGCTGCTGC |
| | CAGTGGCGATAAGTCGTGTCTTACCGGGTTGG |
| | ACTCAAGACGATAGTTACCGGATAAGGCGCAG |
| | CGGTCGGGCTGAACGGGGGGTTCGTGCACACA |
| | GCCCAGCTTGGAGCGAACGACCTACACCGAAC |
| | TGAGATACCTACAGCGTGAGCTATGAGAAAGC |
| | GCCACGCTTCCCGAAGGGAGAAAGGCGGACAG |
| | GTATCCGGTAAGCGGCAGGGTCGGAACAGGAG |
| | AGCGCACGAGGGAGCTTCCAGGGGGAAACGCC |
| | TGGTATCTTTATAGTCCTGTCGGGTTTCGCCA |
| | CCTCTGACTTGAGCGTCGATTTTTGTGATGCT |
| | CGTCAGGGGGGCGGAGCCTATGGAAAAACGCC |
| | AGCAACG |
| SEQ ID NO: 10 | CATGATTTTAAGGGGGTTAGCAGATGCATAAG |
| | TTTAATTTTTTTGTTAAAAAATATTAAACTTT |
| | GTGTTTTTTTTAACAAAATATATTGATAAAAA |
| | TAATAATAGTGGGTATAATTAAGTTGTTAGAG |
| | AAAACGTATAAATTAGGGATAAACTATGGAAC |
| | TTATGAAATAGATTGAAATGGTTTATCTGTTA |
| | CCCCGTATCAAAATTTAGGAGGTTAGTTTAAA |
| | CCTGCAGAGATCTCTCGAGGCGGCCGCGTCGA |
| | CTCTAGACCCGGGAATTCACTGGCCGTCGTTT |
| | TACAACGTCGTGACTGGGAAAACCCTGGCGTT |
| | ACCCAACTTAATCGCCTTGCAGCACATCCCCC |
| | TTTCGCCAGCTGGCGTAATAGCGAAGAGGCCC |
| | GCACCGATCGCCCTTCCCAACAGTTGCGCAGC |
| | CTGAATGGCGAATGGCGCCTGATGCGGTATTT |
| | TCTCCTTACGCATCTGTGCGGTATTTCACACC |
| | GAGCTCACGAAGTCGAGATCAGGGAATGAGTT |
| | TATAAAATAAAAAAAGCACCTGAAAAGGTGTC |
| | TTTTTTTGATGGTTTTGAACTTGTTCTTTTTT |
| | ATCTTGATACATATAGAAATAACGTCATTTTT |
| | ATTTTAGTTGCTGAAAGGTGCGTTGAAGTGTT |
| | GGTATGTATGTGTTTTAAAGTATTGAAAACCC |
| | TTAAAATTGTTTGGACAGAAAAACGCCATGTG |
| | TTAAAGTTATAAGTGACTAAACAAATAACTAA |
| | ATAGATGGGGGTTTCTTTTAATATTATGTGTG |
| | CTAATAGTAGCATTTATTCAGATGAAAAATCA |
| | AGGGTTTTAGTGGACAAGACAAAAAGTGGAAA |
| | AGTGAGACCATGGAGAGAAAAGAAAATCGCTA |
| | ATGTTGATTACTTTGAACTTCTGCATATTCTT |
| | GAATTTAAAAAGGCTGAAAGAGTAAAAGATTG |
| | TGCTGAAATATTAGAGTATAAACAAAATCGTG |
| | AAACAGGCGAAAGAAAGTTGTATCGAGTGTGG |
| | TTTTGTAAATCCAGGCTTTGTCCAATGTGCAA |
| | CTGGAGGAGAGCAATGAAACATGGCATTCAGT |
| | CACAAAAGGTTGTTGCTGAAGTTATTAAACAA |
| | AAGCCAACAGTTCGTTGGTTGTTTCTCACATT |
| | AACAGTTAAAAATGTTTATGATGGCGAAGAAT |
| | TAAATAAGAGTTTGTCAGATATGGCTCAAGGA |
| | TTTCGCCGAATGATGCAATATAAAAAAATTAA |
| | TAAAAATCTTGTTGGTTTTATGCGTGCAACGG |
| | AAGTGACAATAAATAATAAAGATAATTCTTAT |
| | AATCAGCACATGCATGTATTGGTATGTGTGGA |
| | ACCAACTTATTTTAAGAATACAGAAAACTACG |
| | TGAATCAAAAACAATGGATTCAATTTTGGAAA |
| | AAGGCAATGAAATTAGACTATGATCCAAATGT |
| | AAAAGTTCAAATGATTCGACCGAAAAATAAAT |
| | ATAAATCGGATATACAATCGGCAATTGACGAA |
| | ACTGCAAAATATCCTGTAAAGGATACGGATTT |
| | TATGACCGATGATGAAGAAAAGAATTTGAAAC |
| | GTTTGTCTGATTTGGAGGAAGGTTTACACCGT |
| | AAAAGGTTAATCTCCTATGGTGGTTTGTTAAA |
| | AGAAATACATAAAAAATTAAACCTTGATGACA |
| | CAGAAGAAGGCGATTTGATTCATACAGATGAT |
| | GAGGAAAAAGCCGATGAAGATGGATTTTCTAT |
| | TATTGCAATGTGGAATTGGGAACGGAAAAATT |
| | ATTTTATTAAAGAGTAGTTCAACAAACGGGCC |
| | AGTTTGTTGAAGATTAGATGCTATAATTGTTA |
| | TTAAAAGGATTGAAGGATGCTTAGGAAGACGA |
| | GAGATCCTAGCAGCACGCCATAGTGACTGGCG |
| | ATGCTGTCGGAATGGACGATCAAATTCCCCGT |
| | AGGCGCTAGGGACCTCTTTAGCTCCTTGGAAG |
| | CTGTCAGTAGTATACCTAATAATTTATCTACA |
| | TTCCCTTTAGTAACGTGTAACTTTCCAAATTT |
| | ACAAAAGCGACTCATAGAATTATTTCCTCCCG |
| | TTAAATAATAGATAACTATTAAAAATAGACAA |
| | TACTTGCTCATAAGTAACGGTACTTAAATTGT |
| | TTACTTTGGCGTGTTTCATTGCTTGATGAAAC |
| | TGATTTTTAGTAAACAGTTGACGATATTCTCG |
| | ATTGACCCATTTTGAAACAAAGTACGTATATA |
| | GCTTCCAATATTTATCTGGAACATCTGTGGTA |
| | TGGCGGGTAAGTTTTATTAAGACACTGTTTAC |
| | TTTTGGTTTAGGATGAAAGCATTCCGCTGGCA |
| | GCTTAAGCAATTGCTGAATCGAGACTTGAGTG |
| | TGCAAGAGCAACCCTAGTGTTCGGTGAATATC |
| | CAAGGTACGCTTGTAGAATCCTTCTTCAACAA |
| | TCAGATAGATGTCAGACGCATGGCTTTCAAAA |
| | ACCACTTTTTTAATAATTTGTGTGCTTAAATG |
| | GTAAGGAATACTCCCAACAATTTTATACCTCT |
| | GTTTGTTAGGGAATTGAAACTGTAGAATATCT |
| | TGGTGAATTAAAGTGACACGAGTATTCAGTTT |
| | TAATTTTTCTGACGATAAGTTGAATAGATGAC |
| | TGTCTAATTCAATAGACGTTACCTGTTTACTT |
| | ATTTTAGCCAGTTTCGTCGTTAAATGCCCTTT |
| | ACCTGTTCCAATTTCGTAAACGGTATCGGTTT |
| | CTTTTAAATTCAATTGTTTTATTATTTGGTTG |
| | AGTACTTTTTCACTCGTTAAAAAGTTTTGAGA |
| | ATATTTTATATTTTTGTTCATGTAATCACTCC |
| | TTCTTAATTACAAATTTTTAGCATCTAATTTA |
| | ACTTCAATTCCTATTATACAAAATTTTAAGAT |
| | ACTGCACTATCAACACACTCTTAAGTTTGCTT |
| | CTAAGTCTTATTTCCATAACTTCTTTTACGTT |
| | TCCGCCATTCTTTGCTTTTTCGATTTTTATGA |
| | TATGGTGCAAGTCAGCACGAACACGAACCGTC |
| | TTATCTCCCATTATATCTTTTTTTGCACTGAT |
| | TGGTGTATCATTTCGTTTTTCTTTTTATCCCG |
| | CAAGAGGCCCGGCAGTCAGGTGGCACTTTTCG |
| | GGGAAATGTGCGCGGAACCCCTATTTGTTTAT |
| | TTTTCTAAATACATTCAAATATGTATCCGCTC |
| | ATGAGACAATAACCCTGATAAATGCTTCAATA |
| | ATATTGAAAAAGGAAGAGTATGAGTATTCAAC |
| | ATTTCCGTGTCGCCCTTATTCCCTTTTTTGCG |
| | GCATTTTGCCTTCCTGTTTTTGCTCACCCAGA |
| | AACGCTGGTGAAAGTAAAAGATGCTGAAGATC |
| | AGTTGGGTGCACGAGTGGGTTACATCGAACTG |
| | GATCTCAACAGCGGTAAGATCCTTGAGAGTTT |
| | TCGCCCCGAAGAACGTTTTCCAATGATGAGCA |
| | CTTTTAAAGTTCTGCTATGTGGCGCGGTATTA |
| | TCCCGTATTGACGCCGGGCAAGAGCAACTCGG |
| | TCGCCGCATACACTATTCTCAGAATGACTTGG |
| | TTGAGTACTCACCAGTCACAGAAAAGCATCTT |
| | ACGGATGGCATGACAGTAAGAGAATTATGCAG |
| | TGCTGCCATAACCATGAGTGATAACACTGCGG |
| | CCAACTTACTTCTGACAACGATCGGAGGACCG |
| | AAGGAGCTAACCGCTTTTTTGCACAACATGGG |
| | GGATCATGTAACTCGCCTTGATCGTTGGGAAC |
| | CGGAGCTGAATGAAGCCATACCAAACGACGAG |
| | CGTGACACCACGATGCCTGTAGCAATGGCAAC |
| | AACGTTGCGCAAACTATTAACTGGCGAACTAC |
| | TTACTCTAGCTTCCCGGCAACAATTAATAGAC |
| | TGGATGGAGGCGGATAAAGTTGCAGGACCACT |
| | TCTGCGCTCGGCCCTTCCGGCTGGCTGGTTTA |
| | TTGCTGATAAATCTCGAGCCGGTGAGCGTGGG |
| | TCTCGCGGTATCATTGCAGCACTGGGGCCAGA |
| | TGGTAAGCCCTCCCGTATCGTAGTTATCTACA |
| | CGACGGGGAGTCAGGCAACTATGGATGAACGA |
| | AATAGACAGATCGCTGAGATAGGTGCCTCACT |
| | GATTAAGCATTGGTAACTGTCAGACCAAGTTT |
| | ACTCATATATACTTTAGATTGATTTAAAACTT |
| | CATTTTTAATTTAAAAGGATCTAGGTGAAGAT |
| | CCTTTTTGATAATCTCATGACCAAAATCCCTT |
| | AACGTGAGTTTTCGTTCCACTGAGCGTCAGAC |
| | CCCGTAGAAAAGATCAAAGGATCTTCTTGAGA |
| | TCCTTTTTTTCTGCGCGTAATCTGCTGCTTGC |
| | AAACAAAAAAACCACCGCTACCAGCGGTGGTT |
| | TGTTTGCCGGATCAAGAGCTACCAACTCTTTT |
| | TCCGAAGGTAACTGGCTTCAGCAGAGCGCAGA |
| | TACCAAATACTGTTCTTCTAGTGTAGCCGTAG |
| | TTAGGCCACCACTTCAAGAACTCTGTAGCACC |
| | GCCTACATACCTCGCTCTGCTAATCCTGTTAC |
| | CAGTGGCTGCTGCCAGTGGCGATAAGTCGTGT |
| | CTTACCGGGTTGGACTCAAGACGATAGTTACC |
| | GGATAAGGCGCAGCGGTCGGGCTGAACGGGGG |
| | GTTCGTGCACACAGCCCAGCTTGGAGCGAACG |
| | ACCTACACCGAACTGAGATACCTACAGCGTGA |
| | GCTATGAGAAAGCGCCACGCTTCCCGAAGGGA |
| | GAAAGGCGGACAGGTATCCGGTAAGCGGCAGG |
| | GTCGGAACAGGAGAGCGCACGAGGGAGCTTCC |
| | AGGGGGAAACGCCTGGTATCTTTATAGTCCTG |
| | TCGGGTTTCGCCACCTCTGACTTGAGCGTCGA |
| | TTTTTGTGATGCTCGTCAGGGGGGCGGAGCCT |
| | ATGGAAAAACGCCAGCAACGCGGCCTTTTTAC |
| | GGTTCCTGGCCTTTTGCTGGCCTTTTGCTCAC |
| | ATGTTCTTTCCTGCGTTATCCCCTGATTCTGT |
| | GGATAACCGTATTACCGCCTTTGAGTGAGCTG |
| | ATACCGCTCGCCGCAGCCGAACGACCGAGCGC |
| | AGCGAGTCAGTGAGCGAGGAAGCGGAAGAGCG |
| | CCCAATACGCAAACCGCCTCTCCCCGCGCGTT |
| | GGCCGATTCATTAATGCAGGGG |

### EXPERIMENTAL EXAMPLE 2

### Evaluation of Segregational Stability of Shuttle Plasmid (pLK1-MCS)

<2-1> Construction of Transformants

The shuttle plasmid prepared in Experimental Example 1 was introduced into Clostridium acetobutylicum to prepare a transformed recombinant microorganism.

Detailed methods are as follows. Clostridium acetobutylicum was cultured in 100 ml of liquid CGM (Clostridium Growth Medium) (0.75 g/L K₂HPO₄, 0.75 g/L KH₂PO₄, 0.7 g/L, MgSO₄.7H₂O, 0.017 g/L MnSO₄.5H₂O, 0.01 g/L, FeSO₄.7H₂O, 2 g/L (NH₄)₂SO₄, 1 g/L NaCl, 2 g/L asparagine, 0.004 g/L p-aminobenzoic acid, 5 g/L, yeast extract, and 10 g/L glucose) under anaerobic conditions until OD600 reached 1.0. The culture solution was left on ice for 10 minutes, followed by subjecting to centrifugation at 7000 g for 10 minutes at 4° C., thereby obtaining cell pellets. The obtained cell pellets were washed with a buffer solution three times and suspended in 20 ml of the same buffer solution to prepare cells for transformation. To 500 μl of the prepared cells for transformation, 5.0 μg of shuttle plasmids were added, followed by performing electroporation (4 mm cuvette, 2.5 kV, ∞Ω, 25 uF) using a Gene Pulser II prepared by Bio-Rad Corp. Transformed strains were identified in a medium to which erythromycin was added (Table 3).

The plasmids used in the transformation were all methylated in Escherichia coli TOP10 strain transformed with pAN1 plasmid (having genes for methylating inner cytosine in case that GCNGC sequence is present) before electroporation so that the plasmids were not affected by the restriction system of Clostridium acetobutylicum strain.

<2-2> Evaluation of Segregational Stability of pLK1-MCS in Clostridium

The segregational stability of the shuttle plasmid pLK1-MCS was evaluated in Clostridium acetobutylicum strain containing the pLK1-MCS shuttle plasmid constructed in Experimental Example 1. The evaluation was performed by adapting the existing evaluation method (Shin M H, Jung M W, Lee J-H, Kim M D, Kim K H. 2008. Strategies for producing recombinant sucrose phosphorylase originating from Bifidobacterium longum in Escherichia coli JM109. Process Biochemistry 43:822-828).

The shuttle plasmid was introduced into Clostridium acetobutylicum ATCC 824 strain by electroporation, and then cultured in a solid medium containing erythromycin under anaerobic culture conditions at 37° C. for two days. One colony taken from the culture solution was cultured in a culture tube with 40 ml liquid CGM with no antibiotic present at 37° C. until cell concentration (OD600 nm) reached 1.0. The cell concentration was measured using a spectrophotometer (Hach, USA).

The cultured cells were diluted, streaked on solid CGM with no antibiotic present, and cultured at 37° C. for 36 hours. The number of colonies formed was identified. Thereafter, 50 colonies formed were replica plated onto solid CGM containing erythromycin, and the number of cells in which the shuttle plasmid was lost was identified. In case that the shuttle plasmid was lost, colonies could not be formed upon replica plating since there was no erythromycin antibiotic resistance.

Further, 40 ml of liquid CGM with no antibiotic present (diluted to 1/1000 of concentration of the initial culture solution) was inoculated with 40 μL of initial liquid culture solution, and then the aforementioned procedures were repeated 10 times to identify the number of cells in which the shuttle plasmid was lost. The stability from shuttle plasmid loss was evaluated over 100 generations. Since cells were inoculated in a concentration of 1:1000 dilution every time, each generation was assumed to have been divided 10 times (2¹⁰≈1024).

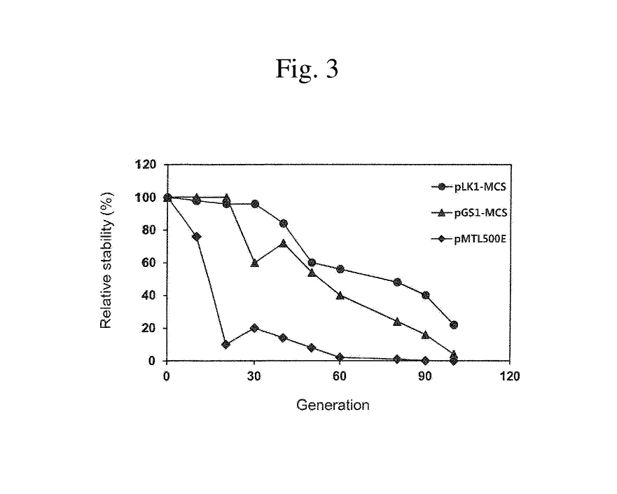

Results are shown in FIG. 3. It can be seen that the novel shuttle plasmid pLK1-MCS showed remarkably improved stability from plasmid loss as compared with the existing pMTL500E (including a replication origin of pAM β1 and a base sequence encoding a replication protein) used as Escherichia coli-Clostridium shuttle plasmid. Further, the novel shuttle plasmid pLK1-MCS was also found to have better segregational stability as compared with pGS1-MCS shuttle plasmid including a replication origin of pIM13 plasmid and a base sequence encoding a replication protein. As a result, the novel shuttle plasmid pLK1-MCS was found to have better segregational stability as compared with the existing shuttle plasmid (Table 3).

TABLE 3

| Plasmid | Number of colony | Temperature (° C.) |
|---|---|---|
| pLK1-MCS | 4.3 × 10² | 37 |
| pMTL500E | 2.6 × 10² | 37 |
| pGS1-MCS | 4.6 × 10² | 37 |

<2-3> Evaluation of Segregational Stability of pLK1-MCS in Escherichia coli

The fact that shuttle plasmid pLK1-MCS constructed in Experimental Example 1 is stably replicated in Escherichia coli was confirmed by transforming the shuttle plasmid pLK1-MCS with Escherichia coli TOP10 containing pAN1 plasmid to obtain methylated pLK1-MCS in Experimental Example <2-1>.

### EXPERIMENTAL EXAMPLE 3

### Expression of Foreign Genes Using Shuttle Plasmids

<3-1> Construction of pLK1-IPA2 Plasmid

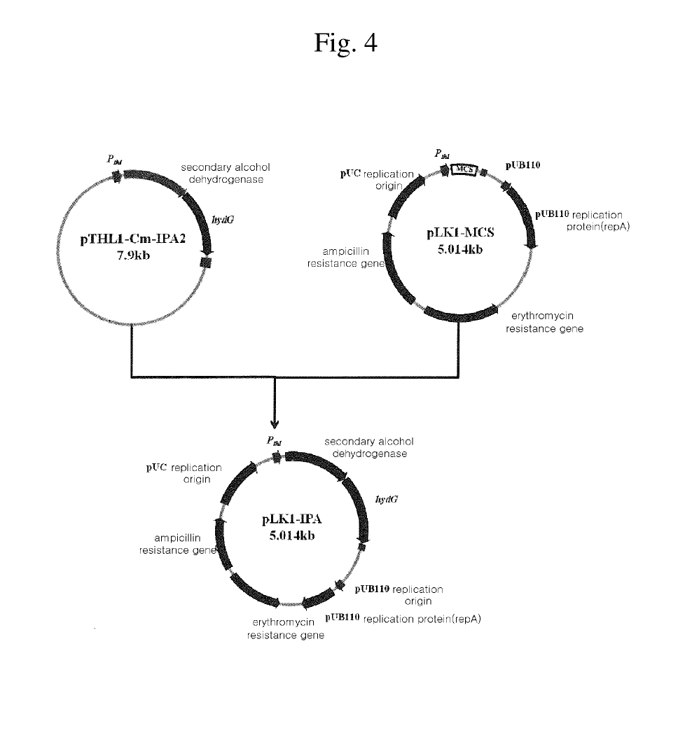

According to Korean Patent Publication No. 10-2011-0032375, it is possible for the secondary alcohol dehydrogenase of Clostridium beijerinckii, NRRL B593 to convert acetone into isopropanol. Accordingly, it was evaluated whether or not the novel shuttle plasmid obtained by recombining the secondary alcohol dehydrogenase gene (IPA-HydG) as a foreign gene to the novel shuttle plasmid of the present invention expresses the secondary alcohol dehydrogenase gene and thus converts acetone into alcohols. A region encoding secondary alcohol dehydrogenase was obtained by PCR reaction using as a template pTHL1-Cm-IPA2 plasmid used in Korean Patent Publication No. 10-2011-0032375 and using as primer base sequences of SEQ ID NO: 11 and SEQ ID NO: 12 (Table 4). The obtained region encoding secondary alcohol dehydrogenase was cloned into the novel plasmid of the present invention, pLK1-MCS, to construct pLK1-IPA2 plasmid. The constructed pLK1-IPA2 plasmid was introduced into Clostridium acetobutylicum PJC4BK strain to obtain a transformed Clostridium acetobutylicum PJC4BK (pLK1-IPA) (FIG. 4).

TABLE 4

| | |
|---|---|
| SEQ ID NO: 11 | CACAGGCCTATGAAAGGTTTTGCAA |
| | TGCTAGGTATTAAT |
| SEQ ID NO: 12 | ATATCTAGATTATTTATCACCTCTG |
| | CAACCACAGCCACC |

In this Experimental Example, the plasmids used in the transformation are all methylated in Escherichia coli TOP10 strain transformed with pAN1 plasmid (having genes for methylating inner cytosine in case that GCNGC sequence is present) before electroporation so that the plasmids were not affected by the restriction system of Clostridium acetobutylicum strain.

<3-2> Identification of Production of Isopropanol Using Clostridium acetobutylicum PJC4BK (pLK1-IPA2)

It was evaluated whether or not the recombined secondary alcohol dehydrogenase was normally expressed to convert acetone into isopropanol in case the recombinant strain prepared in <3-1> is batch cultured.

First, the recombinant Clostridium PJC4BK (pLK1-IPA2) strain prepared in <3-1> of <Experimental Example 3> was streaked on solid CGM, followed by culturing anaerobically at 37° C. overnight. A single colony was inoculated into a 50 ml disposable tube (Falcon, USA) containing 40 ml of CGM, and then cultured anaerobically until OD600 reached 1 at 37° C. . The seed culture was inoculated into 400 ml of CGM containing 1% glucose, followed by standing, and then culturing anaerobically until OD600 reached 1 at 37° C. The 400 ml culture solution was then inoculated into a a fermenter containing 1.6 L of liquid CGM containing 8% glucose. As a control group, Clostridium acetobutylicum PCJ4BK (pTHL-Cm-IPA2) and Clostridium acetobutylicum PCJ4BK were used.

pH was maintained at 5.0 during anaerobic culture using ammonium hydroxide (NH₄OH) and anaerobic conditions were maintained by injecting nitrogen at a speed of 20 ml/min The concentration of the produced butanol and mixed solvent was analyzed every three hours after the initiation of glucose culture. The analysis of butanol and mixed solvent was performed using a gas chromatograph (Agilent, USA). The analysis conditions are as summarized in Table 5.

TABLE 5

| Injector temperature | 320° C. | |
|---|---|---|
| Detector temperature | 320° C. | |
| Injector split ratio | 20/1 | |
| Injection volume | 0.1 | ul |
| Oven condition | 80° C./15 min | |
| Air flow | 300 | mL/min |
| H₂ flow | 30 | mL/min |

| | |
|---|---|
| Column | Supelco CarboWAX |

As a result, it was confirmed that Clostridium acetobutylicum PJC4BK (pLK1-IPA2) had isopropanol producing capability comparable or higher to that of acetobutylicum PJC4BK (pTHL-Cm-IPA2) used as a control group (Table 6).

Consequently, it was confirmed that the shuttle plasmid pLK1-MCS had segregational stability and foreign gene expression capability comparable or higher to those of prior shuttle plasmids.

TABLE 6

| | | Acetone | IPA | Ethanol | Butanol | Total |
|---|---|---|---|---|---|---|
| Strain | Plasmid | (g/L) | (g/L) | (g/L) | (g/L) | (g/L) |
| Clostridium | — | 2.606 | | 2.641 | 15.296 | 20.543 |
| acetobutylicum | | | | | | |
| PJC4BK | | | | | | |
| Clostridium | pTHL- | 0.294 | 4.397 | 3.792 | 15.972 | 24.455 |
| acetobutylicum | Cm-IPA2 | | | | | |
| PJC4BK | | | | | | |
| Clostridium | pLK1- | 0.371 | 4.328 | 3.953 | 16.051 | 24.703 |
| acetobutylicum | IPA2 | | | | | |
| PJC4BK | | | | | | |

Although some embodiments have been described herein, it should be understood by those skilled in the art that these embodiments are given by way of illustration only, and that various modifications, variations, and alterations can be made without departing from the spirit and scope of the invention. Therefore, the scope of the invention should be limited only by the accompanying claims and equivalents thereof.

What is claimed is:

1. A shuttle plasmid replicable in both Clostridium and Escherichia coli comprising:
a base sequence of a first replication origin replicable in Escherichia coli; and
a base sequence of a second replication origin derived from pUB110 plasmid.

2. The shuttle plasmid according to claim 1, further comprising:
a base sequence encoding a replication protein and derived from pUB110 plasmid.

3. The shuttle plasmid according to claim 1, further comprising:
a first antibiotic resistance gene expressed in Escherichia coli.

4. The shuttle plasmid according to claim 1, further comprising:
a second antibiotic resistance gene expressed in Clostridium.

5. The shuttle plasmid according to claim 1, wherein the base sequence of the replication origin derived from pUB110 plasmid is SEQ ID NO: 3.

6. The shuttle plasmid according to claim 2, wherein the base sequence encoding the replication protein region derived from pUB110 plasmid is SEQ ID NO: 4.

7. The shuttle plasmid according to claim 2, wherein the amino acid sequence of the replication protein is SEQ ID NO: 5.

8. A method for producing a transformed microorganism comprising:
preparing a shuttle plasmid according to claim 1; and
introducing the shuttle plasmid into a microorganism.

9. The method according to claim 8, wherein the microorganism is Escherichia coli or Clostridium.

10. The method according to claim 8, wherein, after preparing the shuttle plasmid, a foreign gene is cloned into the shuttle plasmid and the shuttle plasmid into which the foreign gene has been cloned is introduced into a microorganism.

11. A transformed microorganism comprising the shuttle plasmid according to claim 1.

12. The transformed microorganism according to claim 11, wherein the microorganism is Escherichia coli or Clostridium.

13. A method for producing a culture comprising:
culturing the transformed microorganism according to claim 11; and
harvesting a culture.
